(19) United States
(12) Patent Application Publication
(10) Pub. No.: US 2008/0228125 A1
(43) Pub. Date: Sep. 18, 2008

(54) Methods and Devices for Rendering Different Types of Patient Access Devices Compatible with Different Types of Tubing Sets

(75) Inventors: James M. Brugger, Newburyport, MA (US); Jeffrey H. Burbank, Boxford, MA (US); David S. Utterberg, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ProviFlo, LLC A Mississippi corporation, Clinton, MS (US)

(21) Appl. No.: 12/128,547

(22) Filed: May 28, 2008

Related U.S. Application Data
(62) Division of application No. 09/563,674, filed on May 2, 2000.

(51) Int. Cl. A61M 1/14

(52) U.S. Cl. 604/6.16

(57) ABSTRACT

In general, this invention relates to methods and devices for introducing and/or draining fluids into and/or from a patient's body. Methods and devices are provided for rendering conventional tubing sets, or fluid flow sets, which are normally coupleable with only specific complementary patient access devices, compatible with other access devices also. In accordance with one aspect of the invention, methods and devices are provided which render conventional extracorporeal tubing sets to be interchangeably connectable with other specific transcutaneous catheters. In yet another aspect of the invention, non-conventional tubing sets and corresponding non-conventional access devices for use in hemodialysis, or hemofiltration, are provided such that specific tubing sets and access devices can be used interchangeably.

### CROSS-REFERENCES TO RELATED APPLICATIONS

The present application is a divisional patent application claiming priority from U.S. patent application Ser. No. 09/563,674, filed on May 2, 2000, the full disclosure of which is incorporated herein by reference.

This patent application is also related to U.S. patent application Ser. Nos. 08/896,790; 08/896,592; and 08/896,791, all of which were filed on Jul. 18, 1997, the full disclosures of which are incorporated herein by reference.

### FIELD OF THE INVENTION

In general, the present invention is related to methods and devices for introducing and/or draining fluids into and/or from a patient body.

### BACKGROUND OF THE INVENTION

In general, there are two methods whereby a patient's body can be accessed to drain, or withdraw, fluids therefrom, and to deliver fluids thereto. These two methods typically employ patient access devices in the form of catheters or needles respectively.

The catheters are typically transcutaneously implanted on the patient's body and then left in place for extended periods for repeated use, such as to perform more than one treatment. Transcutaneous catheters have distal ends which are typically positioned to be in fluid flow communication with a body cavity, or vessel, or the like. A proximal end of the catheter is then positioned outside the patient body. Fluids are drained, or withdrawn, from the patient body, or delivered to the patient body, by connecting a tubing set to the proximal end of the catheter.

Needles are typically used once only so as to perform a single treatment, and are then discarded. They are typically transcutaneously inserted into a patient body to perform a fluid transfer operation and then withdrawn from the patient body and disposed of after the fluid transfer operation has been completed.

Patients afflicted with end stage renal disease where kidney transplantation is not a viable option, may be treated by hemodialysis or peritoneal dialysis to remove toxic products from the patient's blood. Both techniques operate by the principles of diffusion across semipermeable membranes.

In the case of peritoneal dialysis, the membrane that is used is the patient's peritoneal membrane. In order to perform peritoneal dialysis, a dialysis solution, or dialysate, is delivered into the peritoneal cavity of the patient and caused to remain in the cavity for a dwell period of typically four to six hours, for example. The dialysis solution typically comprises an electrolyte component, to reduce loss of electrolytes, and a sugar component, which acts as an osmotic ingredient to remove water from the patient along with normal metabolic products, such as urea, uric acid, creatinin and/or the like. At the end of the dwell period, spent dialysis solution is drained from the cavity and the cavity is refilled with fresh dialysis solution. Such a dialysate draining and refilling operation is normally performed periodically so as to replace spent dialysate with fresh dialysate.

Conventional peritoneal dialysis systems, currently in use, usually employ patient access devices, in the form of catheters, which are used to access the peritoneal cavity during dialysate draining and refilling operations. Such catheters are normally implanted transcutaneously through the patient's abdomen by a surgeon. When implanted, a distal end of the catheter is positioned to be in flow communication with the peritoneal cavity, and a proximal end of the catheter is positioned outside the patient body. Thereafter, the periodic operation of draining spent dialysate from the peritoneal cavity and refilling the peritoneal cavity with fresh dialysate, is normally performed by the patient.

In conventional dialysis solution delivery systems use is made of a tubing set, often referred to as fluid flow set, to perform a draining and refilling operation. The tubing set is typically connected, or connectable, to a container, containing fresh dialysate, and to an empty container. Such tubing sets can be supplied independently of the containers, or with associated containers pre-mounted thereon so as to form dialysis solution container and tubing set assemblies. To perform the draining and refilling operation, the tubing set is releasably connected to the proximal end of the transcutaneous catheter positioned outside of the patient's body. To this end, complementary connectors are provided on the tubing set and proximal end of the transcutaneous catheter. Accordingly, an external connector connected to the tubing set is coupled to a complementary external connector connected to the transcutaneous catheter. When the connector on the transcutaneous catheter is connected with the complementary connector on the tubing set in this fashion, the spent dialysate is drained from the peritoneal cavity through the transcutaneous catheter and tubing set and into the empty container. When the spent dialysate has been drained from the peritoneal cavity, the peritoneal cavity is replenished with fresh dialysate delivered through the tubing set and transcutaneous catheter to the peritoneal cavity from the other container. Thereafter, the tubing set is disconnected from the transcutaneous catheter, and the transcutaneous catheter is left in place on the patient to permit subsequent draining and refilling operations to be performed in similar fashion.

The peritoneal cavity is particularly susceptible to infection. It has been found that the exterior end of the transcutaneous catheter, protruding from the patient's body, can be a source of contamination of the peritoneal cavity. Consequently, the use of such conventional peritoneal dialysis systems has been rather limited.

A new alternative dialysis solution delivery system, arranged to decrease such risks of contamination, has been developed. The alternative dialysis solution delivery system employs a patient access device in the form of an access tube for accessing a subcutaneously implanted port, instead of a transcutaneous catheter, through which a dialysis solution can be delivered to and/or drained from the peritoneal cavity. The port is arranged transcutaneously to receive any appropriate access tube, such as, for example, a sharp ended needle, a blunt ended needle, a Huber needle, or the like. To access the port, such an access tube is transcutaneously inserted through the patient's skin. Dialysis solution is then selectively delivered to and/or drained from the peritoneal cavity by making use of dialysate containers and an associated tubing set connected to the access tube in a manner similar to that described above.

After a dialysate draining and refilling operation, the access tube is withdrawn from the patient. Accordingly, between dialysate draining and refilling operations, there is an absence of an access device which protrudes from the patient. Consequently the risks of infection of the peritoneal cavity is at least reduced.

A variety of conventional dialysis solution delivery systems are currently available. Such systems typically have manufacturer specific external connectors on the catheters and associated tubing sets respectively. The external connectors of different types of dialysis solution delivery systems are often structurally different. Thus, for example, the external connectors employed by one manufacturer of dialysis solution delivery systems of the transcutaneous catheter type often differ structurally from the external connectors employed by other manufacturers of dialysis solution delivery systems of the transcutaneous catheter type. Therefore, transcutaneous catheters and associated tubing sets made by different manufacturers can normally not be used interchangeably. Consequently, after a specific catheter has been transcutaneously implanted in a patient, the patient is obliged to obtain specific associated tubing sets having external connectors which correspond to the connector of the transcutaneous catheter which has been implanted, so as to enable the periodic draining and refilling operations to be performed by the patient.

It would be advantageous if such conventional tubing sets, or dialysis solution container and tubing set assemblies, having such specific conventional external connectors, could be rendered selectively operable with a subcutaneous port, in addition to the specific transcutaneous catheters for which they are designed. The conventional tubing sets could then be used to access an implanted port as well as their associated catheters. In such a case, a patient would not be limited to obtaining only tubing sets, and/or dialysis solution container and tubing set assemblies, specifically designed to access the subcutaneous port, but would have the option of using currently available tubing sets, and/or dialysis solution container and tubing set assemblies, also. Furthermore, the availability of tubing sets operable with subcutaneous ports would be increased. Should tubing sets associated with access tubes be unavailable, currently available tubing sets, or dialysis solution container and tubing set assemblies, could then be used instead. Stockists, or stockers, of such items, would then not need to increase their inventory to make specific provision for tubing sets associated specifically with access tubes, since the currently available tubing sets would be rendered usable to access a subcutaneous port.

Furthermore, it would be advantageous if tubing sets and associated catheters of the conventional type, could be rendered usable interchangeably. Stockists, or stockers, of such items, would then not need to carry a wide range of different tubing sets, since the currently available tubing sets would be rendered usable interchangeably with a specific implanted transcutaneous catheter.

In the case of hemodialysis, a dialysis operation on a patient's blood is performed outside the patient's body. An artificial semipermeable membrane, as opposed to the peritoneal membrane in the case of peritoneal dialysis, is typically used to perform the dialysis operation. Blood is typically withdrawn from the patient by means of a dialysis machine, and dialysis of soluble substances and water from the blood is performed by diffusion through the artificial semipermeable membrane. After such dialysis, the blood is typically returned to the patient's body.

Traditionally, hemodialysis has been performed by releasably connecting conventional tubing sets of a hemodialysis machine with complementary access devices, such as transcutaneous catheters extending from the patient's body, so as to perform a hemodialysis operation. Conventional connectors, in the form of a 6% taper male and female Luer type connectors, are used to connect the tubing sets with the access devices. The connectors used have specific internal diameters. It has been found that if connectors having internal diameters greater than the internal diameters of the conventional connectors were to be used, higher blood flow rates could be achieved during a hemodialysis operation. The time taken to perform an average hemodialysis operation could then be reduced and patient discomfort would be decreased. However, should non-conventional tubing sets and non-conventional catheters, having connectors with such greater internal diameters, be introduced for use in hemodialysis, compatibility between the conventional tubing sets and catheters currently in use, and such non-conventional tubing sets and catheters, can arise. For example, it could happen that a user ends up with a conventional tubing set with its associated connector, and a non-conventional transcutaneous catheter with its associated connector, and, consequently, would then be unable to connect the one with the other to perform a hemodialysis operation. It could also happen that a user ends up with a conventional transcutaneous catheter with its associated connector and a non-conventional tubing set with its associated connector, and, consequently, would then also be unable to connect the one with the other to perform a hemodialysis operation.

Accordingly, it would be advantageous if tubing sets and associated access devices having connectors with relatively large internal diameters could be introduced for use in hemodialysis, thereby to enable higher blood flow rates to be achieved during a hemodialysis operation. Furthermore, it would be advantageous if such non-conventional tubing sets and associated access devices, having connectors with relatively large internal diameters, could be introduced such that they are not only connectable with each other, but with the conventional tubing sets and access devices currently in use in hemodialysis also. In such a case, compatibility problems between such non-conventional hemodialysis access devices and tubing sets, having the relatively large internal diameter connectors, and the conventional hemodialysis tubing sets and access devices currently in use, would be avoided.

It will be appreciated that a hemofiltration procedure is normally performed in a fashion similar to hemodialysis. The tubing sets and access devices used in hemofiltration are typically similar to those used in hemodialysis. Accordingly, it is to be appreciated that, where appropriate, whenever the term hemodialysis is used in this specification, it should be interpreted to extend to hemofiltration as well. Furthermore, although the invention will be described with reference to its application in the fields of peritoneal dialysis, hemodialysis and hemofiltration, it is to be appreciated that the application of the invention is not to be limited to these fields only, but extends to the field of delivering and/or draining fluids into and/or from a patient body in general.

### SUMMARY OF THE INVENTION

Accordingly, it is an object of this invention to provide access tube sets, or needle sets, arranged to be connectable to specific connectors of conventional tubing sets, which specific connectors are arranged normally to be coupled with specific complementary connectors on transcutaneous catheters, thereby to render such conventional tubing sets coupleable with subcutaneous ports.

It is another object of this invention to provide access tube sets, or needle sets, which are arranged to be connectable to specific connectors on conventional tubing sets currently used in peritoneal dialysis, so as to render such conventional tubing sets coupleable with subcutaneous ports.

It is yet another objective of this invention to render different types of conventional tubing sets and associated catheters currently used in peritoneal dialysis compatible with one another, so as to enable such different tubing sets and associated catheters to be usable interchangeably.

It is yet another object of this invention to provide non-conventional tubing sets and associated access devices for use in hemodialysis, which tubing sets and associated access devices have complementary connectors with internal diameters larger than the internal diameters of the connectors currently used on conventional hemodialysis tubing sets and associated access devices.

It is yet a further object of this invention to provide non-conventional tubing sets and associated access devices for use in hemodialysis, which tubing sets and associated access devices have complementary connectors with internal diameters larger than the internal diameters of the connectors currently used on conventional hemodialysis tubing sets and associated access devices, and which non-conventional tubing sets and access devices are not only connectable with each other, but with conventional tubing sets and access devices currently used in hemodialysis also.

According to one aspect of the invention, there is provided a method of fluidically communicating between a patient's body and an extracorporeal tubing set. the method comprises providing an extracorporeal tubing set having a fluid transfer tube defining opposed ends, an access tube for selectively connecting one end of the fluid transfer tube percutaneously to a subcutaneously implanted port, and an external connector for selectively connecting the one end of the fluid transfer tube extracorporeally to a transcutaneous catheter.

According to another aspect of the invention, there is provided a method of fluidically communicating between a patient's body and at least two different extracorporeal tubing sets. The method comprises providing an access device for accessing the patient's body, the access device having a distal end portion transcutaneously positionable in the patient's body and an opposed proximal end, a first connector for connecting the proximal end selectively to a connector on a first tubing set, and a second connector for connecting the proximal end selectively to a different connector on another tubing set.

According to another aspect of the invention, there is provided a method of fluidically communicating between a patient's body and at least two different patient access devices. the method comprises providing a tubing set comprising a fluid flow tube having a distal end and an opposed proximal end, the tubing set further comprising a first connector, for connecting the distal end of the fluid flow tube selectively to a connector on one access device, and a second connector, for connecting the distal end of the fluid flow tube selectively to a different connector on another access device.

According to yet another aspect of the invention, there is provided an extracorporeal tubing set. The extracorporeal tubing set comprises at least one fluid transfer tube for transferring a fluid to or from a patient body, said tube having at least a proximal end and a distal end, wherein the proximal end is operatively connected, or connectable, to an extracorporeal instrument, medicament, or receptacle. the tubing set further comprises an access tube operatively connected, or connectable, to the distal end of the fluid transfer tube, said access tube being percutaneously connectable to an implanted port, and an external connector operatively connected, or connectable, to the distal end of the fluid transfer tube, said external connector being externally connectable to a transcutaneous catheter.

According to another aspect of the invention, there is provided a dialysis tubing set comprising a fluid transfer tube having a proximal end and a distal end, wherein the proximal end is connectable to a source of dialysis solution, an access tube connected, or connectable, to the distal end of the fluid transfer tube, said access tube percutaneously connectable to an implanted port, and an external connector connected, or connectable, to the distal end of the fluid transfer tube, said external connector externally connectable to a transcutaneous catheter.

According to a further aspect of the invention, there is provided an access tube set for adapting at least one tubing set, having an external connector normally connectable to a specific complementary connector of a transcutaneous catheter, so as to render the tubing set operatively connectable with an implanted subcutaneous port. The access tube set comprises an access tube connectable with the implanted subcutaneous port, and a connector operatively connected to the access tube, the connector being complementary to the external connector of the tubing set, so that the connector of the access tube set can be connected to the external connector of the tubing set thereby to render the tubing set selectively connectable to the implanted port.

According to another aspect of the invention, there is provided an adapter kit for adapting any one of a plurality of different tubing sets, each having a specific external connector normally connectable to a specific complementary external connector of a transcutaneous catheter, so as to render any one of the plurality of different tubing sets selectively connectable with an implanted subcutaneous port. The adapter kit comprises an access tube set having an access tube connectable with the implanted subcutaneous port and a connector operatively connected to the access tube, the connector being complementary to the external connector of a specific one of the different tubing sets so that the access tube set can be connected to that external connector to render that tubing set operatively connectable to the implanted port. The adapter kit further comprises at least one adapter comprising a first connector, releasably connectable to the connector of the access tube set, and an opposed connector connected to the first connector, the opposed connector being complementary to the external connector of another specific one of the different tubing sets, the adapter being mountable on the connector of the access tube set thereby to enable the access tube set to be connected to the external connector of the other specific one of the different tubing sets thereby to render the other specific one of the different tubing sets operatively connectable to the implanted port.

According to a further aspect of the invention, there is provided an access tube set comprising an access tube arranged to access a patient body, and at least two different connectors operatively connected, or connectable, to the access tube, each connector being complementary to a different conventional connector of a different conventional tubing set, so as to render the access tube selectively connectable to any one of the different conventional tubing sets.

According to another aspect of the invention, there is provided an access tube set comprising an access tube arranged to access a patient body, and at least two different connectors operatively connected, or connectable, to the access tube, each connector being complementary to a different conventional connector of a different conventional tubing set, so as to render the access tube selectively connectable to any one of the different conventional tubing sets.

According to another aspect of the invention, there is provided an access device, for accessing a patient body to perform a fluid transfer procedure, the access device comprising an accessing portion arranged to access a patient body, and at least two connectors operatively connected, or connectable, with the accessing portion, each connector being complementary to a different connector of different tubing sets so as to render the access device selectively coupleable to any one of the different tubing sets.

### BRIEF DESCRIPTION OF THE DRAWINGS

The invention will now be described, by way of example, with reference to the accompanying diagrammatic drawings, in which:

FIG. 1 shows a schematic diagram indicating a new dialysis solution delivery system which includes a dialysis solution container, containing dialysate, and an empty container, for receiving spent dialysate, the containers being releasably coupleable to a subcutaneous port by means of an access tube so as selectively to deliver dialysis solution to, and to drain spent dialysis solution from, a peritoneal cavity in a patient's body;

FIG. 2 shows a schematic diagram of an alternative access tube arrangement for use in the dialysis solution delivery system of FIG. 1;

FIG. 3 shows a schematic diagram of another alternative access tube arrangement for use in the dialysis solution delivery system of FIG. 1;

FIGS. 4A to 4C show diagrams schematically indicating three different conventional dialysis solution delivery systems, each including a dialysis solution container assembly releasably coupleable to an associated transcutaneous catheter by means of external connectors, so as to deliver fresh dialysis solution to, and to drain spent dialysis solution from, a peritoneal cavity in a patient's body;

FIGS. 4D to 4F correspond to FIGS. 4A to 4C, but show only the tubing sets of the different conventional dialysis solution delivery systems, containers of the dialysis solution delivery systems having been removed;

FIGS. 5A to 5C show schematic diagrams of access tube sets, or needle sets, in accordance with the invention, for rendering the conventional tubing sets shown in FIGS. 4A to 4F compatible with the subcutaneous port shown in FIG. 1;

FIG. 6 shows a schematic side view of part of a tubing set, in accordance with the invention, the tubing set being selectively coupleable to a conventional transcutaneous catheter and the implanted port shown in FIG. 1;

FIG. 6A shows a schematic side view of the tubing set, shown in FIG. 6, being used to access the subcutaneous port of FIG. 1;

FIG. 6B shows a schematic side view of the tubing set, shown in FIG. 6, being used with a conventional transcutaneous catheter;

FIG. 7 shows a schematic side view of part of another tubing set, in accordance with the invention;

FIG. 8 shows a schematic side view of part of yet another tubing set, in accordance with the invention;

FIG. 9 shows a schematic side view of part of yet another tubing set, in accordance with the invention;

FIG. 10 shows a schematic diagram of an adapter kit in accordance with the invention;

FIG. 11 shows a schematic diagram of part of another adapter kit in accordance with the invention;

FIG. 12 shows a schematic diagram of the rest of the kit shown in FIG. 11;

FIG. 12A shows a schematic diagram of part of a tubing set and adapter assembly in accordance with the invention;

FIG. 13 shows a schematic side view of an access tube set, or needle set, in accordance with the invention, and which can be used in hemodialysis;

FIG. 14 shows a schematic side view of the needle set of FIG. 13 being used to render a non-conventional, relatively high flow hemodialysis tubing set, compatible with a subcutaneous port, so that a hemodialysis operation can be performed using the port and such a non-conventional tubing set;

FIG. 15 shows a schematic side view of the needle set of FIG. 13 being used to render a conventional, standard flow hemodialysis tubing set, compatible with a subcutaneous port, so that a hemodialysis operation can be performed using the port and a conventional tubing set;

FIG. 16 shows a schematic side view of a non-conventional catheter arrangement for use in hemodialysis, the catheter arrangement having adapters to enable it to be selectively coupled to either corresponding non-conventional hemodialysis tubing sets, or conventional hemodialysis tubing sets;

FIG. 16A shows a schematic side view of a priming connector for use with the needle set of FIG. 13 or the non-conventional catheter of FIG. 16;

FIG. 17 shows a schematic side view of a non-conventional hemodialysis tubing set having an adapter releasably mounted thereon, the adapter being arranged to enable the tubing set to be connected selectively to a corresponding non-conventional transcutaneous catheter, or needle set, so as to perform a hemodialysis operation using a non-conventional transcutaneous catheter, or needle set, or to a conventional transcutaneous catheter, so as to perform a hemodialysis operation using a conventional transcutaneous catheter; and

FIG. 18 shows a schematic side view of a priming connector for use with the tubing set of FIG. 17.

### DESCRIPTION OF THE SPECIFIC EMBODIMENTS

The present invention will now be described in an application to delivering and draining a dialysis solution to and from a peritoneal cavity in a patient body. Thereafter, the invention will be described in an application to hemodialysis and hemofiltration.

As mentioned, peritoneal dialysis typically requires periodic draining of spent dialysis solution from the peritoneal cavity and replacement of the spent dialysis solution with fresh dialysis solution. The dialysis solution, or dialysate, typically comprises a solution which will promote diffusion or osmosis across a patient's peritoneal membrane, so as to remove toxic by-products from the patient's blood. In particular forms of peritoneal dialysis, such as Continuous Ambulatory Peritoneal Dialysis (CAPD) and Continuous Cycling Peritoneal Dialysis (CCPD), the dialysate, after initial delivery into the peritoneal cavity, remains in the cavity for a dwell period of usually 4 to 6 hours. During this time, the dialysate removes normal metabolic products such as urea, uric acid, creatinin, and/or the like, from the patient's body, by osmosis through the peritoneal membrane. At the conclusion of the dwell period, the used or spent dialysate is drained from the peritoneal cavity and typically replaced by a new supply of unused or fresh dialysate.

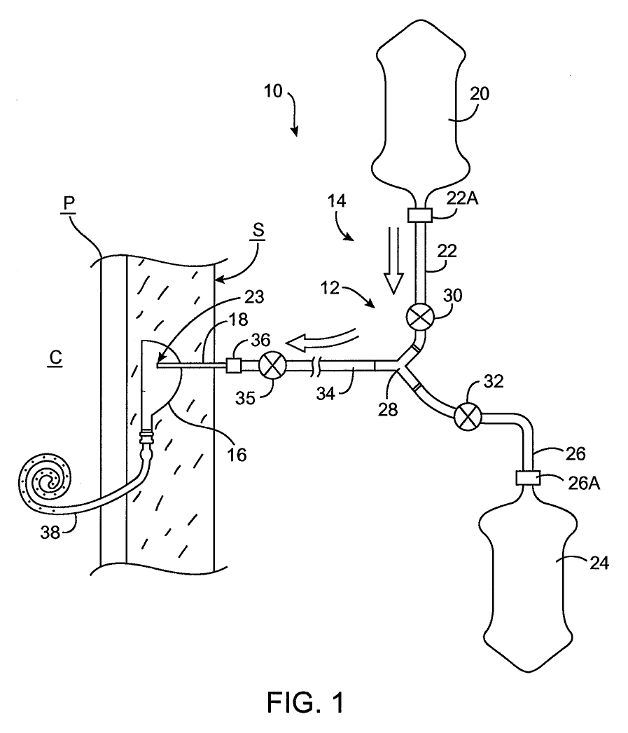

Referring to FIG. 1, a preferred new peritoneal dialysis solution delivery system is indicated generally by reference numeral 10. The system 10 includes a subcutaneous port, generally indicated by reference numeral 16, and a dialysis solution container and tubing set assembly, generally indicated by reference numeral 14.

The dialysis solution container and tubing set assembly 14 includes containers 20, 24 and a tubing set, sometimes referred to as a fluid flow set, generally indicated by reference numeral 12. The tubing set 12 includes an access tube, schematically indicated in simplified form at 18, typically in the form of a needle, for selectively accessing the implanted subcutaneous port 16. Container 20 is for delivery of dialysis solution to the peritoneal cavity, and, accordingly, is normally filled with fresh dialysate. Container 20 is connected in fluid flow communication with a first fluid transfer tube 22 of tubing set 12. The container 20 can be releasably coupled to the tube 22 by means of a coupling 22A. Container 24 is for receiving spent dialysis solution, and, accordingly, is normally empty. Container 24 is connected in fluid flow communication with a second fluid transfer tube 26 of tubing set 12. The container 24 can be releasably coupled to the tube 26 by means of a coupling 26A. Containers 20 and 24 are typically of a flexible material such as a polymer, or synthetic plastics material, or the like. The first tube 22 and the second tube 26 are typically connected to a junction at 28. A fluid flow controller 30 on tube 22 and a similar controller 32 on tube 26 can be provided to regulate dialysate flow from and to the containers 20 and 24. The fluid flow controllers 30, 32 can comprise clamps, or the like, for example. The fluid flow controllers 30, 32 can be used alternately to block fluid flow to or from the containers 20, 24, or to increase or decrease dialysate flow through the tubes 22, 26.

The access tube 18 extends from a single fluid transfer tube 34 which is coupled in fluid flow communication to junction 28. A further fluid flow controller can be provided at 35. Access tube 18 typically has a relatively large bore to permit relatively high flow rates of dialysate therethrough. Access tube 18 can be in the form of any appropriate access tube, such as, for example, a large bore coring needle, a blunt ended needle, a fistula needle, a Huber needle, or the like. By “coring needle” is meant a non-Huber type access tube. Such a coring needle can have a distal tip 23 defining a sharp or blunt end capable of penetrating tissue in a forward direction so as to engage with port 16. Instead, access tube 18 can be in the form of a needle having a non-coring design, such as a Huber needle, which has a side-facing distal opening. Access tube 18 typically has an outer diameter of at least 1.6 mm (16 G). Instead, it can have an outer diameter of at least 1.83 mm (15 G), or at least 2.13 mm (14 G), or at least 13 G. It can have a bore size as large as 12 G, or larger. The access tube 18 can be made of any suitable material, such as, stainless steel or surgical steel, a hard plastics material, or the like.

Optionally, a connector 36, such as a Luer type connector, can be provided, so as to render access tube 18 detachably connected to the tube 34 to permit access tube replacement, thereby to inhibit contamination by using the same access tube more than once if the tubing set 12 is used more than once.

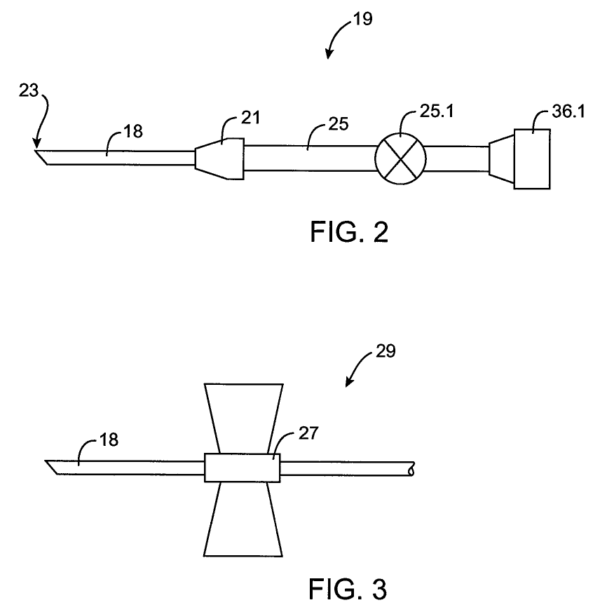

Referring to FIG. 2, in which like reference numerals are used to designate similar parts unless otherwise stated, an alternative access tube arrangement to that shown in FIG. 1, is generally indicated by reference numeral 19. The access tube arrangement 19 comprises a connector 36.1, typically in the form of a conventional female Luer type connector, for releasable connection to a complementary male Luer type connector at 36 in FIG. 1. It further includes a hub 21 to which access tube 18 is connected. A tube 25 having a fluid flow controller 25.1 extends between the hub and the connector 36.1. Whenever reference is made to access tube 18 in this specification, access tube arrangement 19 can be used instead, where appropriate.

Referring to FIG. 3, instead of the access tube 18, a Fistula type needle 29 can be used. The fistula type needle 29 comprises an access tube 18 mounted on a winged hub 27. For the purposes of this specification, where reference is made to an access tube 18, a fistula needle 29 can be used instead, where appropriate.

Referring again to FIG. 1, the port 16 is typically implanted subcutaneously by a surgeon, and a catheter 38, attached to port 16, is positioned to extend between port 16 and the peritoneal cavity C so as to define a flow passage between the port 16 and the peritoneal cavity C. The port 16 is typically implanted subcutaneously in the patient so that the catheter 38 extends into the peritoneal cavity C of the patient's peritoneum P while port 16 is positioned beneath the patient's skin S.

In use, fresh dialysate is initially introduced into the peritoneal cavity C by transcutaneously accessing the port 16 with the access tube 18, and then permitting fresh dialysis solution to flow or drain from the container 20, through access tube 18, along the fluid flow passage defined by catheter 38, and into the peritoneal cavity C. This can be achieved by holding container 20 at an elevation above the peritoneal cavity C to permit the dialysate to gravitate from the container 20 into the peritoneal cavity C. After the dialysate is delivered in this way, access tube 18 is withdrawn from port 16.

After a certain period, e.g., four to six hours, typically another system 10 is used to drain the now spent dialysis solution from the peritoneal cavity C and to deliver fresh dialysate into the cavity C. Access tube 18, is inserted through the skin S of the patient and into the port 16. Typically, the spent dialysate is permitted to drain or gravitate from the peritoneal cavity into the container 24. Fluid flow controller 30 is typically in a closed condition when the spent dialysate is drained into container 24 to prevent fluid contact between fresh dialysate in container 20 and the spent dialysate draining from the peritoneal cavity C into container 24. The draining of the spent dialysate from the peritoneal cavity C is typically achieved by holding container 24 at an elevation below the peritoneal cavity C to permit the dialysate to gravitate from the peritoneal cavity C and into container 24.

After the spent dialysate has been drained, fresh dialysate is caused to flow or gravitate from container 20 through the catheter 38 and into the peritoneal cavity C in a fashion similar to that described above. This is typically achieved by closing fluid flow controller 32 and opening fluid flow controller 30 to inhibit contamination of the fresh dialysate by the spent dialysate. Once the delivery of fresh dialysate into the peritoneal cavity C of the patient has been completed, access tube 18 is removed from the patient. After a period of typically four to six hours, the above operation is repeated.

Although the system 10 can be reused by refilling container 20 with fresh dialysate and disposing of the spent dialysate in container 24, for hygienic purposes, such a procedure is not normally followed. The tubing set 12 can be retained for subsequent use and containers 20 and 24 replaced with fresh containers by removing the used containers from the couplings 22A, 26A and replacing them with fresh containers. An unused, or fresh, access tube 18 can then be connected to the tubing set 12 by detaching the used access tube 18 from the connector 36 and replacing it with such an unused access tube. Such a procedure is also not normally followed. Instead, each time a draining and refilling operation of the peritoneal cavity C is performed, use is made of a fresh dialysis solution container and tubing set assembly 14, in which container 20 is pre-filled with fresh dialysate and container 24 is empty to receive spent dialysate from the peritoneal cavity C. The containers 20, 24 and the tubing set 12, can typically be supplied separately, in which case the containers 20, 24 are connected to the tubing set 12 by the patient prior to a dialysate draining and refilling operation. Instead, complete dialysis solution container and tubing set assemblies 14 can be provided, in which case containers 20, 24 are supplied together with tubing set 12. In view of the importance of hygiene and the susceptibility of the peritoneal cavity to infection, such fresh dialysis solution container and tubing set assemblies 14, or containers 20 and 24 and tubing sets 12, are typically supplied in packages and in a sterile condition.

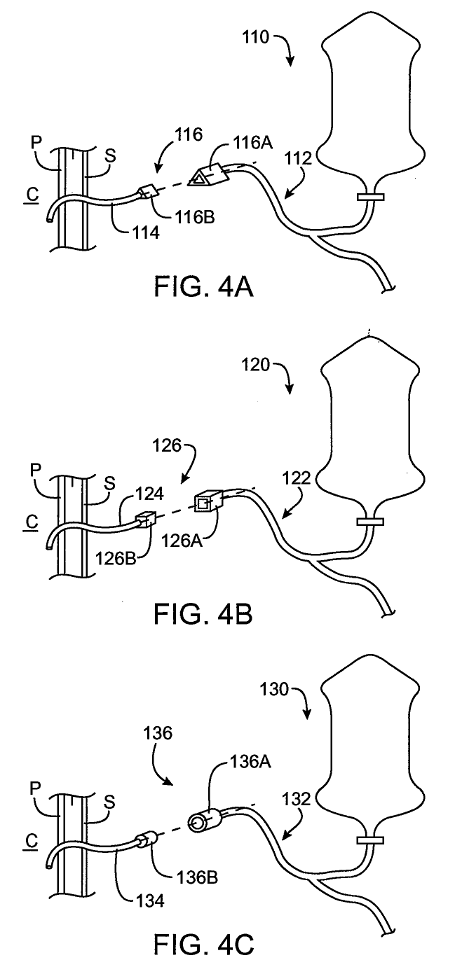

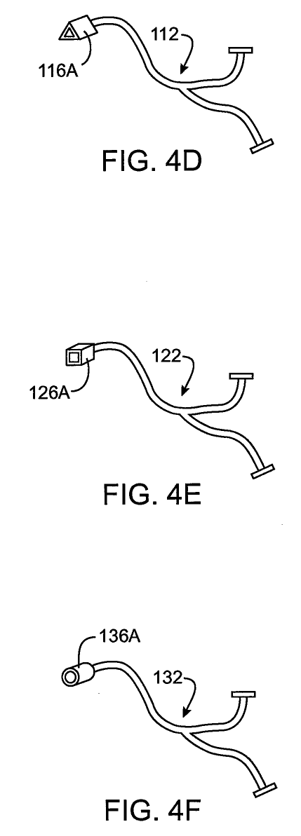

Referring now to FIGS. 4A, 4B, and 4C of the drawings, three schematic representations of different conventional dialysis solution delivery systems, which are currently in use, are generally indicated by reference numerals 110, 120 and 130, respectively. The dialysis solution delivery systems 110, 120, 130 can represent any of those currently available, such as those available from Baxter, Fresnius, Gambro, JMS, Terumo, or B. Braun, for example. The systems 110, 120 and 130 function in similar fashion to the system 10. Each system 110, 120 and 130 includes a dialysis solution container and tubing set assembly comprising a tubing set 112, 122, 132, and associated containers, similar to the containers 20, 24 in FIG. 1. The containers for receiving spent dialysate from the patient are not shown in FIGS. 4A, 4B and 4C. The tubing sets 112, 122, 132 are arranged for extracorporeal connection to transcutaneous catheters 114, 124, 134, respectively. The catheters 114, 124, 134 are typically implanted by a surgeon and subsequent dialysis solution draining and refilling operations are typically performed by the patient. The catheters 114, 124, 134 are typically in the form of transcutaneously implanted catheters extending between the peritoneal cavity and a position outside the patient's body. The tubing sets 112, 122, 132 are releasably coupleable with their specific associated transcutaneous catheters 114, 124, 134, by means of complementary specific external connectors generally indicated at 116, 126, 136. The external connectors at 116, 126, 136 include an external connector 116A, 126A, 136A connected to be in fluid flow communication with the tubing sets 112, 122, 132, respectively, and complementary external connectors 116B, 126B, 136B connected on proximal ends of the catheters 114, 124, 134, to be in fluid flow communication with the transcutaneous catheters 114, 124, and 134.

The systems 110, 120, and 130 are schematic representations of available dialysis solution systems. Each system functions in similar fashion. However, the external connectors at 116, 126, and 136 differ. In FIGS. 4A, 4B, and 4C, the different external connectors at 116, 126, and 136 are schematically indicated as generally having cross-sectionally triangular, rectangular, and circular shapes for illustrative purposes only, so as to indicate that the external connectors of the different types of systems 110, 120, 130 currently available, are structurally different, and are normally not usable interchangeably.

Referring to FIGS. 4D to 4F, the tubing sets 112, 122, and 132 are shown without their associated containers. The tubing sets 112, 122, 132 can be used repeatedly, the containers then being replaced selectively between dialysate draining and refilling operations. Normally, however, the tubing sets 112, 122, 132 are discarded after use and fresh tubing sets are used each time a dialysate draining and refilling operation is performed. The tubing sets 112, 122, 132 and their associated containers 20, 24 can be supplied separately, so that the containers 20, 24 are connected to the tubing sets 112, 122, 132 by the patient prior to performing a dialysate draining and refilling operation. Instead, dialysis solution container and tubing set assemblies can be supplied, in which case the containers are supplied together with the tubing sets.

In accordance with one aspect of the invention, an access tube set, or needle set, is provided, which renders the currently available conventional peritoneal tubing sets, such as those represented and schematically indicated by reference numerals 112, 122, and 132 in FIGS. 4A to 4F, compatible with a subcutaneous port 16 as shown in FIG. 1 of the drawings.

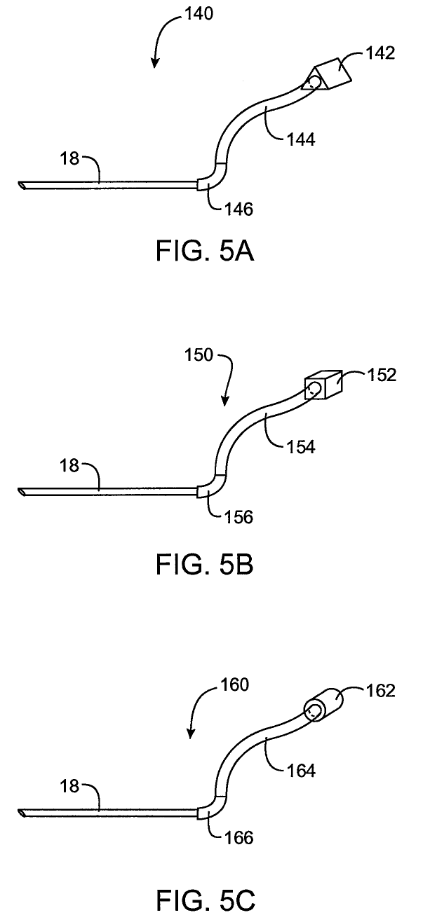

Referring now to FIGS. 5A, 5B, and 5C of the drawings, reference numerals 140, 150 and 160 schematically indicate three such access tube, or needle sets, in accordance with the invention, for use with peritoneal tubing sets 112, 122, and 132 respectively, so as to serve as adapters to render the tubing sets 112, 122, 132 compatible with the subcutaneous port 16. Each needle set 140, 150, and 160 includes an access tube 18, similar to the access tube 18 of FIG. 1. Each of the respective access tubes 18 is connected in fluid flow communication with an associated connector 142, 152, 162. Connector 142 is complementary to the external connector 116A of tubing set 112, connector 152 is complementary to the external connector 126A of tubing set 122, and connector 162 is complementary to the external connector 136A of tubing set 132. The access tubes 18 are typically connected to their associated connectors 142, 152, and 162 by means of tubes 144, 154, and 164. The tubes can be made of a flexible material such as a synthetic or natural rubber, a plastics material, Polyvinyl Chloride (PVC), or the like. Preformed bends, 146, 156, and 166 can be provided to direct tubes 144, 154, and 164 to lie adjacent a patient's skin when the port 16 is accessed by access tubes 18.

The needle sets 140, 150, 160, can each be provided separately, typically in packages and in a sterile condition. A patient having an implanted port 16, can then obtain an appropriate needle set 140, 150, 160, so as to render a corresponding conventional tubing set 112, 122, 132 compatible with the implanted port 16.

In accordance with another aspect of the invention, tubing sets 112, 122, 132, or the container and tubing set assemblies 110, 120, 130, can be provided with an associated needle set 140, 150, and 160, respectively, to render such tubing sets, or assemblies, selectively connectable to the conventional complementary connectors of transcutaneous catheters 114, 124, 134, and, in addition, to the port 16 as shown in FIG. 1. Such tubing sets, and/or assemblies, having such needle sets, could typically be supplied in packages and in a sterile condition. Tubing set 112, or assembly 110, for example, can typically be packaged in a condition in which its associated needle set 140 is in a pre-mounted condition, connector 142 then being coupled to external connector 116A. Accordingly, if the patient has a transcutaneous catheter 114, needle set 140 can then be detached from external connector 116A and discarded. Tubing set 112 can then be coupled to catheter 114 directly in conventional fashion. If, however, the patient has an implanted port 16, needle set 140 is retained in its mounted condition on external connector 116A so that tubing set 112 can be coupled to port 16 by transcutaneously engaging access tube 18 with port 16. Each of the tubing sets 122, 132, or assemblies 120, 130 can be prepackaged in a condition in which their associated needle sets 150, 160 are mounted on the respective external connectors 126A, 136A, in a similar fashion.

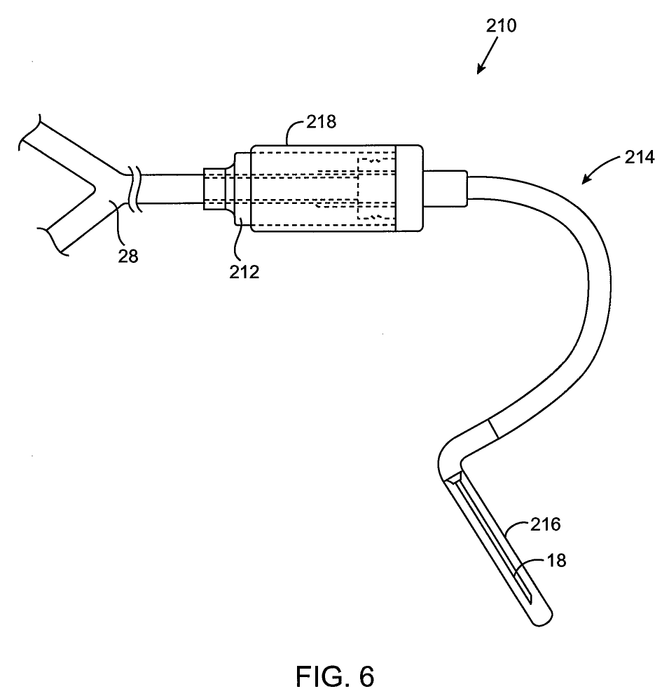

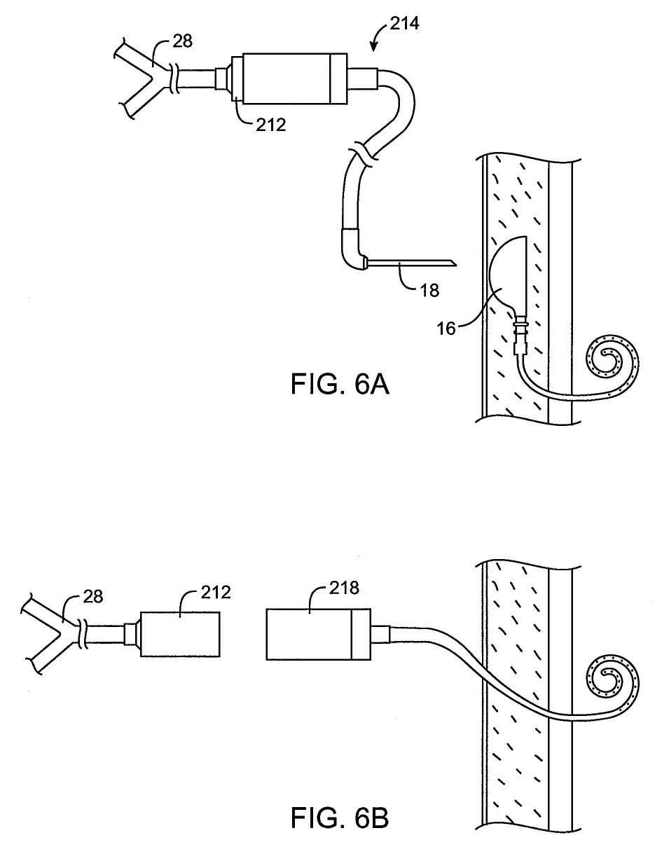

An example of a conventional dialysis tubing set, having an associated needle set pre-mounted thereon, in accordance with the invention, to render it selectively coupleable to an implanted port or a conventional corresponding transcutaneous catheter, is generally indicated by reference numeral 210 in FIG. 6 of the drawings.

In FIG. 6, the same reference numerals have been used to designate similar parts unless otherwise indicated. Containers (not shown in FIG. 6), similar to the containers 20, 24 in FIG. 1, are typically connected in fluid flow communication with junction 28, in use. As in the case of tubing sets 112, 122, and 132, the tubing set of FIG. 6 has a specific conventional external connector 212. External connector 212 can correspond with any one of external connectors 116A, 126A, 136A shown in FIGS. 4A to 4F. A needle set 214, in accordance with the invention, is shown in a pre-mounted condition on external connector 212. The needle set 214 includes a connector 218 complementary to external connector 212 and which can correspond with any one of connectors 142, 152, 162 shown in FIGS. 5A to 5C depending on the connector 212. The access tube 18 of needle set 214 is shrouded in a detachable cap 216 to inhibit accidental injury when handled. As mentioned, the tubing set, having its associated needle set 214 pre-mounted thereon, is typically packaged in a sterile condition.

Referring now to FIGS. 6A and 6B of the drawings, the tubing set 210 will now be described in use. If the patient has a pre-implanted port 16, as shown in FIG. 6A, tubing set 210 is simply removed from its package, cap 216 is removed from access tube 18, and tube 18 is transcutaneously inserted into subcutaneous port 16 to perform a dialysis solution delivery and/or drainage operation, as previously described. If the patient has a corresponding transcutaneous catheter having a conventional external connector 218 complementary to connector 212, needle set 214 is simply detached from connector 212, and tubing set 210 is used in conventional fashion as indicated in FIG. 6B. In FIG. 6B, needle set 214 is shown as having been removed and discarded.

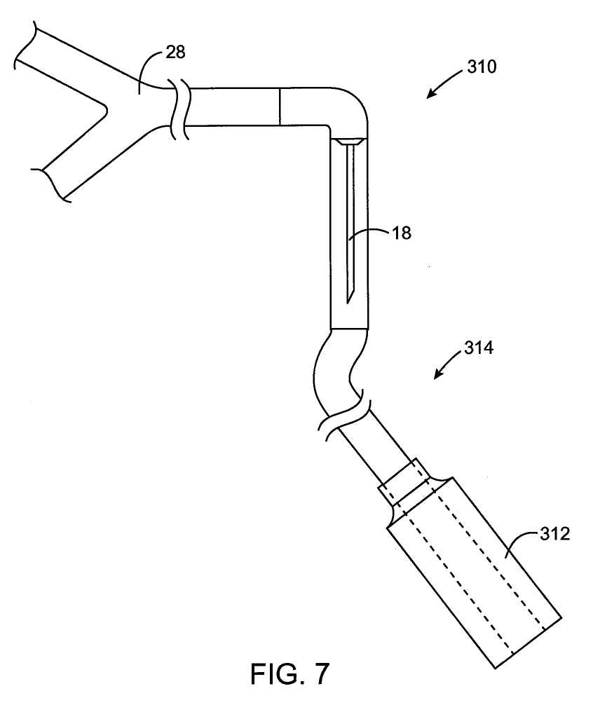

Referring now to FIG. 7 of the drawings, another tubing set, in accordance with the invention, is generally indicated by reference numeral 310. The tubing set 310 is similar to tubing set 210, save that its external connector 312 is positioned forwardly of its access tube 18. Thus, tubing set 310 has an adapter 314 detachably coupled to access tube 18, the specific external connector 312 being carried at a free end of adapter 314. In use, when a patient has a corresponding conventional transcutaneous catheter with an external connector corresponding with connector 312, tubing set 310 is used with adapter 314 in a mounted condition. Should the patient have a subcutaneous port, adapter 314 is detached to expose access tube 18. After such detachment, access tube 18 can be used to access the subcutaneous port.

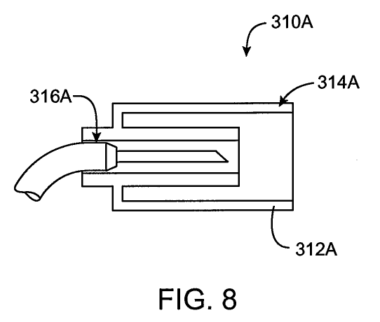

FIG. 8 shows yet another tubing set, in accordance with the invention, generally indicated by reference numeral 310A. The tubing set 310A is similar to the tubing set 310, save that the adapter 314A of tubing set 310A is releasably and directly mounted relative to the access tube 18 without an intervening tube. The conventional external connector 312A is carried on an end portion of a tube of the tubing set as indicated at 316A. If the patient has a corresponding transcutaneous catheter, tubing set 310A is used with adapter 314A mounted on the tubing set as shown in FIG. 8. The adapter 314A is removed to expose, or reveal, the access tube 18 for use with a subcutaneous port, should the patient have such a subcutaneous port and not a transcutaneous catheter.

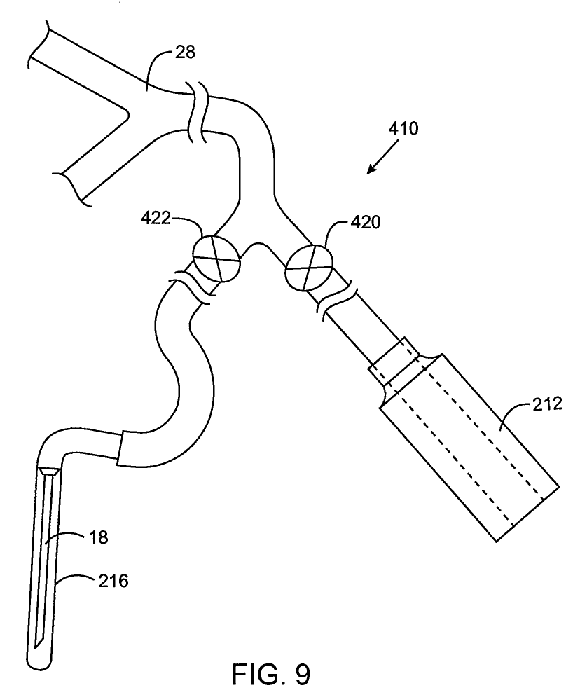

Referring now to FIG. 9 of the drawings, yet another tubing set, in accordance with the invention, is generally indicated by reference numeral 410. Tubing set 410 includes a conventional external connector 412 and an access tube 18 connected in parallel and in fluid flow communication with the junction 28. Flow controllers 420 and 422 are provided selectively to block passage of dialysate. In use, the appropriate one of access tube 18 and external connector 412 is selected to access the port or corresponding conventional transcutaneous catheter, depending on which one is implanted in the patient. Depending on which one of the access tube 18 and the connector 412 is selected, the controllers 420, 422 are used to block flow through the non-selected one of the access tube 18 and the connector 412 and to permit flow through the selected one of the access tube 18 and connector 412.

External connectors 212, 312, 312A, 412, can be complementary to any one of the conventional types of connectors currently in use to perform peritoneal dialysis operations.

According to another aspect of the invention, the needle, or access tube sets, 140, 150, and 160 of FIGS. 5A to 5C of the drawings, can be supplied together as a kit. Accordingly, an appropriate needle set can then be selected from the kit to render either one of tubing sets 112, 122, or 132 compatible with the subcutaneous port 16 shown in FIG. 1. The needle sets 140, 150, 160 can be designed for re-use. Instead, they can be designed to be disposable. The needle sets 140, 150, 160 of the kit can advantageously be supplied in a common package, and in a sterile condition.

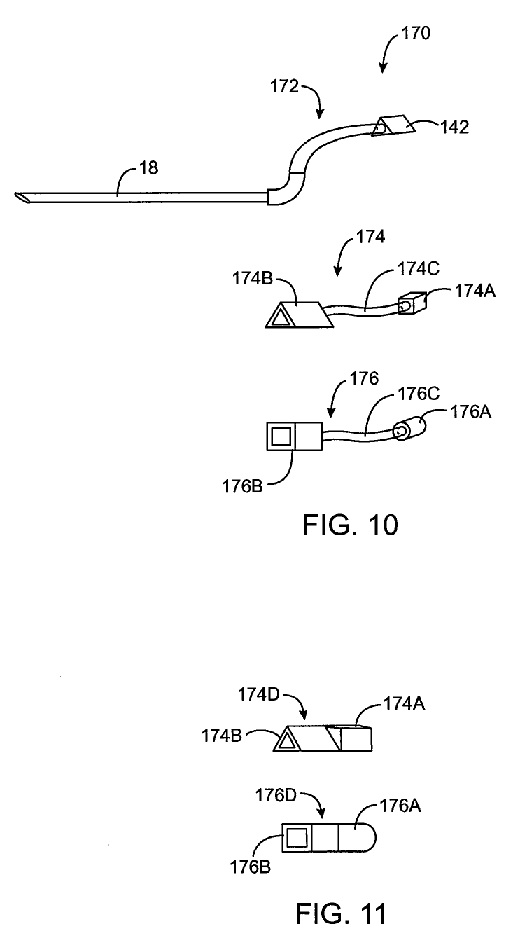

Referring now to FIG. 10 of the drawings, another needle set kit, in accordance with the invention, is generally indicated by reference numeral 170. Kit 170 includes a needle set 172 which can be similar to any one of the needle sets 140, 150, and 160 described above with reference to FIGS. 5A to 5C. In this case needle set 172 is similar to needle set 140. Accordingly, needle set 172 includes a connector 142 complementary to currently available external connector 116A. If the tubing set to be adapted has an external connector 116A complementary to connector 142, the connector 142 can be coupled to external connector 11 6A as described above to render that dialysis solution tubing set, or dialysis solution container and tubing set assembly, compatible with port 16.

Kit 170 further includes a plurality of adapters 174, 176, of which only two are shown schematically by way of example. It will be appreciated that any number of such adapters can be supplied with the kit to widen the range of currently available tubing sets to be rendered compatible with port 16. Each adapter 174, 176 has a connector 174A, 176A which is complementary to a specific type of currently available tubing set connector. The adapters have opposed connectors 174B, 176B. One of the opposed connectors, in this case connector 174B, is complementary to connector 142 so as to be releasably coupleable therewith. The opposed connector 176B of the other adapter is complementary to connector 174A of adapter 174. Accordingly, the adapters 174, 176 can be connected one to another in series on the connector 142.

Although adapters 174, 176 are shown as having tube portions 174C, 176C, it will be appreciated that, instead, the connectors 174A, 176A can be connected directly to the opposed connectors 174B, 176B as indicated by reference numerals 174D and 176D in FIG. 11. Advantageously, the kits of FIGS. 10 and 11, and the kit described with reference to FIGS. 5A to 5C, can be pre-packaged in a sterile condition.

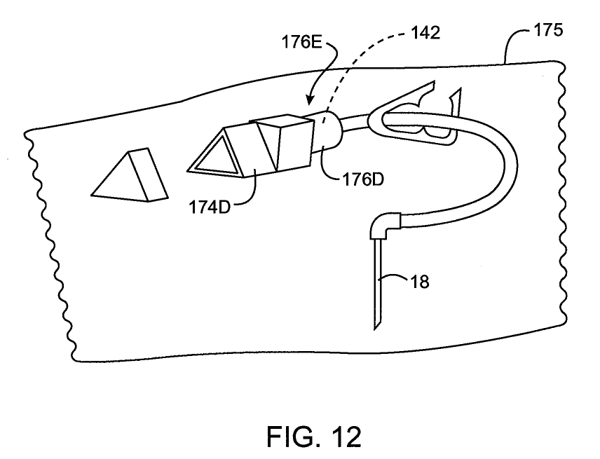

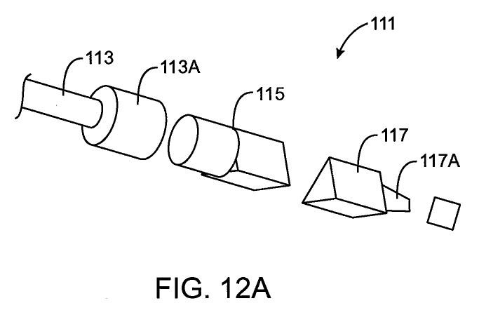

In use, needle set 172 and the adapters 174, 176, or 174D, 176D, can be connected one to another in series so as to define an adapter chain, as indicated at 176E in FIG. 12. Thus, to render a specific conventional tubing set, or dialysis solution container and tubing set assembly, compatible with port 16, the chain can be arranged, or adapters removed from an end of the chain remote from the access tube 18, so that an appropriate connector, which is complementary to the external connector of the tubing set being used, is at the end. Such an assembly, or kit, as indicated in FIG. 12, can typically be packaged in a package or container 175 in a sterile condition.

In one embodiment, for example, the connector 142 can be complementary to a Baxter type connector. A single adapter, similar to the adapter 174D can be provided. Such an adapter can have opposed ends in which a connector 174B, complementary to the Baxter type connector, is defined at its one end, for releasable connection to a Baxter type connector 142. Another type of connector 174A, such as a Fresnius type of connector, can then be defined at the opposed end of the adapter 174D. Conveniently, a cap can be provided for releasable connection to the Fresnius type connector when the adapter is not in use. Such a kit can then be used selectively to adapt a tubing set carrying a Baxter type external connector or a tubing set carrying a Fresnius type external connector for use with a subcutaneous port.

Instead, adapters 174, 176, or 174D, 176D, can each have an opposed connector coupleable with connector 142, shown in FIG. 10, so that a single adapter can be selected and used to couple needle set 172 to an external connector of a specific conventional tubing set.

Referring to FIG. 12A, and in accordance with another aspect of the invention, a tubing set and adapter assembly is generally indicated by reference numeral 111. The tubing set and adapter assembly 111 comprises a tubing set 113 and adapters 115, 117. The tubing set 113 comprises a conventional connector 113A corresponding to any one of the specific conventional connectors currently used on tubing sets of conventional dialysis solution delivery systems. The adapters 115, 117 are releasably connectable to one another in a stacked condition on connector 113A to render the tubing set 113 selectively coupleable to a plurality of different complementary specific connectors currently used on conventional transcutaneous catheters such as those provided by different manufacturers. Accordingly, the adapters 115, 117 can be used in a fashion similar to the adapters 174D, 176D shown in FIG. 11, but in this case, to render the tubing set 113 compatible with a plurality of different conventional catheters, such as those available from different manufacturers. One of the adapters, namely adapter 117, can have a connector, indicated at 117A, coupleable to a needle set, such as those indicated in FIGS. 1 to 3, to render the tubing set usable with such a needle set so as to access an implanted port.

In this manner, tubing sets 112, 122, 132, or the container and tubing set assemblies 110, 120, 130, can be provided with associated adapters, to render such tubing sets, or assemblies, selectively connectable to the conventional complementary connectors of transcutaneous catheters 114, 124, 134, and, in addition, to the port 16 as shown in FIG. 1. Such tubing sets, and/or assemblies, having such adapters, could typically be supplied in packages and in a sterile condition. Tubing set 112, or assembly 110, for example, can typically be packaged in a condition in which its associated adapters are in a pre-mounted condition, in a stacked condition, on external connector 116A. Accordingly, if the patient has a transcutaneous catheter 114, the adapters can then be detached from external connector 116A and discarded. Tubing set 112 can then be coupled to catheter 114 directly in conventional fashion. If, however, the patient has another type of conventional transcutaneous catheter, having a different type of specific conventional connector, such as connector 126B, 136B, or an implanted port 16, the adapters can be arranged to enable the tubing set 112 to be connected thereto. It will be appreciated that each of the tubing sets 122, 132, or assemblies 120, 130, can be prepackaged in a condition in which associated adapters are mounted on the respective external connectors 126A, 136A, in a similar fashion.

The application of the invention in the field of hemodialysis will now be described. Hemofiltration is performed utilizing tubing sets and access devices similar to those used in hemodialysis. Accordingly, even though the invention will now be described with reference to hemodialysis, it will be appreciated that the invention applies to hemofiltration as well.

Traditionally, hemodialysis has been performed using specific, conventional, connectors, to connect an extracorporeal hemodialysis tubing set to a patient access device, such as a transcutaneous catheter, so as to perform a hemodialysis operation. The conventional connectors which have been used to date are in the form of male and female Luer type connectors having internal diameters of a specific size. The male Luer type connectors are normally mounted on the hemodialysis tubing set and the female Luer type connectors are normally mounted on the access device, used to access the patient. As mentioned, it has been found that should non-conventional connectors, having larger internal diameters than the conventional Luer type connectors which are currently being used, be used instead, hemodialysis procedures would be enhanced by enabling higher blood flow rates to be achieved. Since the conventional tubing sets and access devices are often supplied separately, it would be advantageous to provide such non-conventional hemodialysis tubing sets and access devices having such larger internal diameter connectors such that the non-conventional hemodialysis tubing sets and access devices can be used with the conventional hemodialysis access devices and tubing sets as well. In such a case, compatibility problems between the conventional and non-conventional hemodialysis tubing sets and access devices would be avoided.

An access tube set, or needle set, which can be used advantageously in hemodialysis, and/or hemofiltration, in accordance with another aspect of the invention, will now be described with reference to FIGS. 13 to 15 of the drawings. In FIGS. 13 to 15, like reference numerals are used to designate similar parts unless otherwise stated.

Referring to FIG. 13, the needle set is generally indicated by reference numeral 510. The needle set 510 is arranged selectively to enable a conventional hemodialysis, or hemofiltration, tubing set, having a conventional connector in the form of a male Luer type connector, or a non-conventional hemodialysis, or hemofiltration, tubing set, having a non-conventional connector, which has an internal diameter greater than that of the conventional Luer connectors, to be coupled to an implanted port. Accordingly, the needle set 510 includes an access tube 18. The access tube 18 is arranged to engage a subcutaneous port, similar to the subcutaneous port 16 shown in FIG. 1, so as to enable such a subcutaneous port to be used to perform a hemodialysis, or hemofiltration, procedure. Instead, the access tube can be arranged for direct access to a patients vasculature to perform a hemodialysis, or hemofiltration, procedure, in which case, it can be in the form of a conventional needle, or the like.

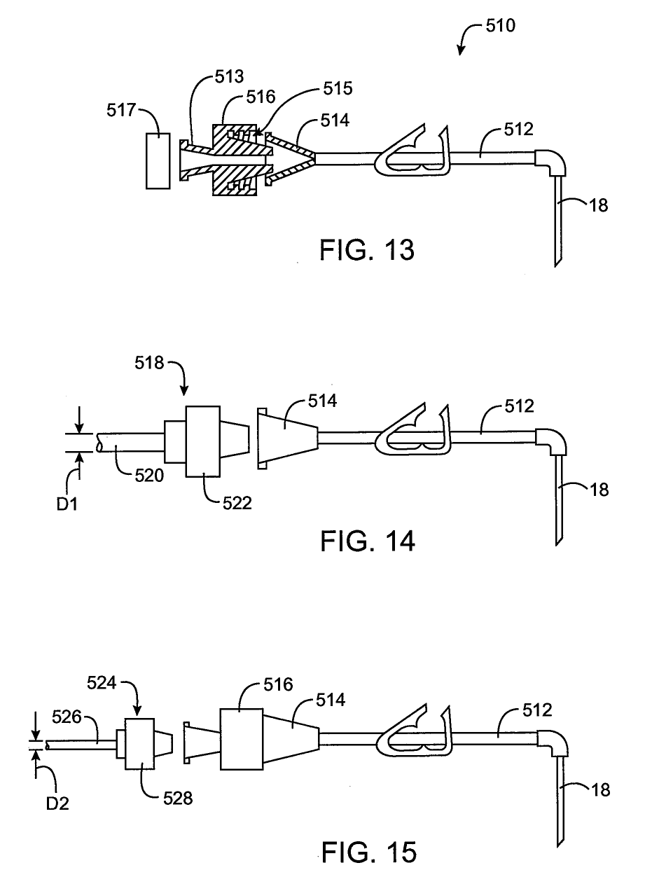

The access tube 18 is mounted at an end of a tube 512. A non-conventional connector 514 is connected to, typically bonded with, an opposed end of the tube 512. The connector 514 can be in any appropriate form, but has an internal diameter greater than the internal diameter of the conventional Luer type connectors currently used in hemodialysis and hemofiltration procedures. Conveniently, the connector 514 is in the form of a Luer, or Luer-like, female connector 514. The connector 514 is arranged to be releasably connectable to a complementary non-conventional connector on a non-conventional tubing set of a hemodialysis, or hemofiltration, machine. Naturally, if the connector 514 is in the form of a Luer, or Luer-like female connector, the corresponding connector on the non-conventional tubing set would be in the form of a complementary Luer, or Luer-like, male connector. Such a complementary Luer, or Luer-like, male connector, of such a non-conventional hemodialysis, or hemofiltration, tubing set, is indicated by reference numeral 522 in FIG. 14. A conventional male Luer type connector currently used in hemodialysis, and hemofiltration procedures, is indicated by reference numeral 528 in FIG. 15. The conventional connectors are of the I.S.O. 594/1 and 594/2 type. The non-conventional connectors 514, 522 can be in the form of I.S.O. 8637, connectors, for example.

Referring again to FIG. 13, the needle set 510 further includes an adapter 516 releasably mounted on the non-conventional connector 514. In the case where the connector 514 is in the form of a Luer, or Luer-like female connector, the adapter 516 comprises a complementary Luer, or Luer-like, male connector indicated by reference numeral 515. The adapter 516 further includes a conventional female Luer connector 513, corresponding to the conventional male Luer type connector 528 currently used in hemodialysis and hemofiltration procedures. When the adapter 516 is mounted on the non-conventional connector 514 the needle set 510 can be connected to a conventional male Luer type connector on a conventional hemodialysis tubing set. Advantageously, a cap 517, such as a conventional male Luer type cap, releasably mountable on the conventional female Luer connector 513 can be provided. The cap 517 is typically mounted on the female Luer connector 513 when the needle set 510 is not in use, thereby, for example, to inhibit foreign matter from fouling the needle set 510.

With reference to FIG. 14, use of the needle set 510 in a relatively high blood flow hemodialysis, or hemofiltration, application, will now be described. In such a high flow hemodialysis application, a non-conventional extracorporeal tubing set, part of which is indicated generally by reference numeral 518, is operatively connected to a hemodialysis, or hemofiltration, machine (not shown) in conventional fashion. The tubing set 518 typically includes a tube 520 having an internal diameter D1, which is typically larger than an internal diameter D2 of a tube of a conventional hemodialysis, or hemofiltration, tubing set 524 as shown in FIG. 15. However, the diameter D1 need not necessarily be larger than the diameter D2, since it has been found that higher blood flow rates can be achieved by providing connectors with relatively large internal diameters only.

The tube 520 is connected to the non-conventional connector 522. Where a non-conventional tubing set 518 is used, the needle set 510 can be used to render the tubing set 518 compatible with a subcutaneous port 16, similar to the one shown in FIG. 1. To use the needle set 510, the adapter 516 is removed from the rest of the needle set 510 to expose the connector 514. The connector 514 of the needle set 510 is then connected to the connector 522 as indicated in FIG. 14. The access tube 18 is then connected in fluid flow communication with the non-conventional tubing set 518 by means of the non-conventional connectors 514, 522 so as to enable higher flow rates to be achieved. The access tube 18 can then be used to access a subcutaneous port in a manner similar to that already described above, so as to perform a relatively high blood flow rate hemodialysis, or hemofiltration, procedure.

Referring now to FIG. 15, where a conventional tubing set 524 is being used, the needle set 510 can be used to render also such a conventional tubing set 524 coupleable with an implanted port. In such a case, the conventional extracorporeal tubing set 524, is typically operatively connected to a hemodialysis, or hemofiltration, machine (not shown). The tubing set 524 typically includes the tube 526 and the conventional Luer type male connector 528. To use the needle set 510, the cap 517 is dismounted from the conventional female Luer connector 513 and the female Luer connector 513 is then connected to the conventional male Luer connector 528 in conventional fashion. The access tube 18 can then be used to access an implanted port to perform a hemodialysis, or hemofiltration, operation.

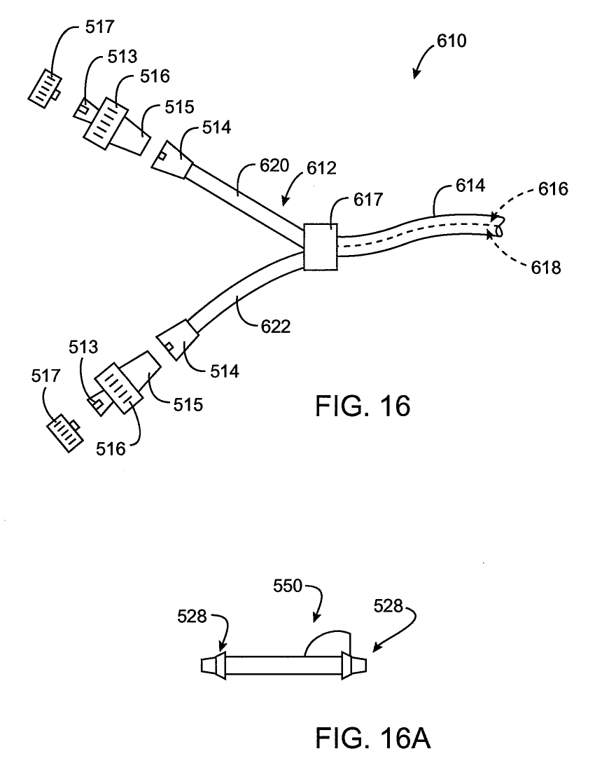

Although in conventional hemodialysis and hemofiltration procedures, the conventional male Luer type connector is normally on the tubing set and the corresponding conventional female Luer type connector is on the access device, such as the transcutaneous catheter, this need not be the case. Instead, the conventional male Luer type connector can be on the access device and the corresponding conventional female Luer type connector can be on the tubing set. In such a case, a conventional male Luer type connector can be used on the adapter 516 instead of the female Luer connector 513. Instead, the needle set 510 can be provided with a priming connector 550 shown in FIG. 16A. The priming connector 550 defines opposed conventional male type Luer connectors 528. The priming connector can be releasably mounted on the conventional female Luer connector 513. Accordingly, should the tubing set have a conventional female Luer type connector, the priming connector can be retained in a mounted condition on the connector 513 so as to render the needle set 510 connectable to the conventional female Luer type connector on the tubing set. Should the tubing set have a conventional male Luer type connector, the priming connector 550 is simply removed.

After the needle set 510 has been used, it is typically discarded. Accordingly, it is typically a disposable item.

The needle set described above with reference to FIGS. 13 to 15 provides a needle set which renders a hemodialysis, or hemofiltration, tubing set, whether the tubing set is of a conventional type, or of a non-conventional type which provides for higher blood flow rates, compatible with a subcutaneous port. When the hemodialysis tubing set being used has a non-conventional connector having a larger internal diameter, and the needle set is used as indicated in FIG. 14, blood flow rates greater than that in conventional hemodialysis operations can be achieved.

In accordance with yet another aspect of the invention, a hemodialysis, or hemofiltration, catheter arrangement, which can be used selectively with non-conventional tubing sets, each of which is similar to the tubing set 518 shown in FIG. 14, or with conventional tubing sets, each of which is similar to the tubing set 524 in FIG. 15, will now be described with reference to FIG. 16.

In FIG. 16, in which like reference numerals have been used to designate similar parts, unless otherwise stated, an access device, in the form of a catheter arrangement, which can advantageously be used to access a patient to perform a hemodialysis, or hemofiltration, procedure, is generally indicated by reference numeral 610. The arrangement 610 comprises a catheter, generally indicated by reference numeral 612. The catheter 612 comprises a catheter portion 614, which is transcutaneously insertable into a patient's body so as to access his or her vasculature. The catheter portion 614 defines two longitudinally extending lumens 616, 618 respectively. In use, blood is withdrawn from the patient's body through one of the lumens 616, 618, and the blood is returned to the patient's body through the other of the lumens 616, 618.

The catheter 612 further comprises two tubular portions 620, 622. The tubular portions 620, 622 are operatively connected to the catheter portion 614 at a junction 617, such that the lumen 616 extends longitudinally along tubular portion 620, and the lumen 618 extends longitudinally along the tubular portion 622.

A non-conventional connector 514 is mounted on a free end of each of the tubular portions 620, 622. As described above, with reference to FIGS. 13 to 15, the connectors 514, 514 can be in any appropriate form, but having internal diameters greater than that of the conventional Luer connectors currently used in hemodialysis and hemofiltration procedures, so as to provide for higher blood flow rates. Conveniently, by way of example, the connectors 514, 514 can be in the form of female Luer, or Luer-like, connectors.

An adapter 516, similar to the adapter described above with reference to FIGS. 13 and 15, is provided on each of the connectors 514, 514. Accordingly, each adapter 516, 516 has a connector 515 releasably mountable on the connectors 514, 514. Each adapter 516, 516 further comprises an opposed female Luer type connector 513, 513 which corresponds to the conventional Luer connectors currently used in hemodialysis and hemofiltration, procedures. Caps 517, 517 releasably mounted on the connectors 513, 513 can also be provided.

Use of the arrangement 610, in a relatively high blood flow hemodialysis, or hemofiltration, application, will now be described. In such a relatively high flow application, a non-conventional extracorporeal tubing set, similar to that indicated by reference numeral 518 in FIG. 14, is operatively connected to an inlet and an outlet respectively of a hemodialysis, or hemofiltration, machine (not shown) in conventional fashion. To use the arrangement 610, the catheter portion 614 is introduced into a patient body to be in flow communication with the patient's vasculature. The adapters 516, 516 are then removed from the connectors 514, 514 to expose the connectors 514, 514. The connectors 514, 514 are then connected to connectors, each of which is similar to the connector 522 indicated in FIG. 14, which connectors are mounted on the tubing sets 518, 518 connected to the inlet and outlet of the hemodialysis, or hemofiltration, machine. A hemodialysis, or a hemofiltration, procedure, can then be performed at a blood flow rate greater than that normally achieved in conventional hemodialysis and hemofiltration procedures since the connectors 514, 522 have internal diameters larger than the internal diameters of the conventional Luer connectors currently used to perform such procedures.

Use of the arrangement 610 with conventional hemodialysis, or hemofiltration, tubing sets, will now be described. Each of the conventional tubing sets is similar to the tubing set shown in FIG. 15 and indicated by reference numeral 524. The tubing sets 524, 524 are typically operatively connected to an inlet and an outlet of a hemodialysis, or hemofiltration, machine (not shown). The tubing sets 524, 524 have conventional male Luer type connectors 528, 528. To use the arrangement 610, the caps 517, 517 are dismounted from the conventional female Luer connectors 513, 513 and the female Luer connectors 513, 513 are then connected to the conventional male Luer connectors 528, 528 in conventional fashion. A hemodialysis, or a hemofiltration, procedure, can then be performed in conventional fashion.

A priming connector 550, as shown in FIG. 16A, can be provided on the connectors 513, 513 to render the arrangement 610 selectively coupleable to conventional tubing sets having conventional female Luer type connectors.

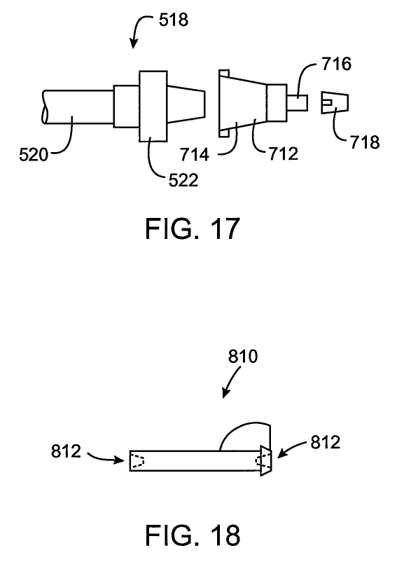

A non-conventional hemodialysis, or hemofiltration, tubing set arrangement, in accordance with another aspect of the invention, which can be used advantageously in hemodialysis, and/or hemofiltration, procedures, will now be described with reference to FIG. 17.

The non-conventional tubing set arrangement is generally indicated by reference numeral 710 and comprises a non-conventional tubing set 518 similar to the tubing set 518 indicated in FIG. 14. Accordingly, the tubing set 518 comprises a non-conventional connector 522 having an internal diameter greater than that of the conventional Luer type connectors currently used in hemodialysis and hemofiltration, so as to provide for greater blood flow rates. As mentioned above, the connector 522 can be in any appropriate form. Conveniently, and by way of example only, the connector is in the form of a Luer, or Luer-like, male connector. The connector 522 is mounted on, or bonded with, an end of the tube 520. An opposed end of the tube 520 is connected, or connectable, to an inlet, or an outlet, of a hemodialysis, or hemofiltration, machine, in conventional fashion.

The tubing set arrangement 710 further comprises an adapter 712. The adapter 712 includes a connector 714 complementary to the connector 522. In the case where the connector 522 is in the form of a non-conventional Luer, or Luer-like, male connector, the connector 714 is in the form of a complementary Luer, or Luer-like, female connector. The adapter 712 further comprises a conventional male Luer type connector 716, which corresponds to the conventional Luer type connectors currently used in hemodialysis, and hemofiltration, procedures.

The tubing set arrangement 710 can conveniently be supplied together with a hemodialysis, or hemofiltration, machine, or can be supplied separately for mounting on a hemodialysis, or hemofiltration, machine. Conveniently, the tubing set arrangement 710 can be supplied with the adapter 714 mounted on the connector 522. A cap 718, releasably mounted on the connector formation 716 can be provided to inhibit fouling of the tubing set arrangement when not in use.

In use, the tubing set arrangement 710 is operatively connected to a hemodialysis, or hemofiltration, machine, in conventional fashion. In the case where an access device having a conventional Luer type female connector is to be used to access the patient to perform a hemodialysis, or hemofiltration, procedure, the adapter 712 is retained in a mounted condition on the connector 522. The cap 718 is then removed to expose the conventional male Luer type connector 716. The connector 716 can then be connected to the conventional access device in conventional fashion so as to access the patient to perform a hemodialysis, or hemofiltration, procedure.

In the case where a non-conventional access device, having a non-conventional connector, such as the connector 514 shown in FIGS. 13 to 15, is to be used to access the patient, the adapter 712 is removed from the tubing set 518 so as to expose the connector 522. The connector 522 is then connected to the connector 514 to access the patient and to perform a hemodialysis, or hemofiltration, procedure.

Although the arrangement 710 can be formed to be reusable, it is preferably of a disposable type. Thus, after a procedure has been performed, the arrangement 710 is typically discarded.

A priming connector 810, shown in FIG. 18, can be provided on connector 716. The priming connector has opposed conventional female Luer connectors 812, 812 of the type conventionally used in hemodialysis and hemofiltration procedures. Accordingly, should the access device have a conventional male connector, the priming connector 810 can be used to render the tubing set arrangement 710 coupleable thereto.

Although the foregoing invention has been described in some detail by way of illustration and example, for purposes of clarity of understanding, it will be obvious that certain changes and modifications may be practiced within the scope of the appended claims.

What is claimed is:

1. An extracorporeal tubing set comprising:
at least one fluid transfer tube for transferring a fluid to or from a patient body, said tube having at least a proximal end and a distal end, wherein the proximal end is operatively connected, or connectable, to an extracorporeal instrument, medicament, or receptacle;
an access tube operatively connected, or connectable, to the distal end of the fluid transfer tube, said access tube being percutaneously connectable to an implanted port; and
an external connector operatively connected, or connectable, to the distal end of the fluid transfer tube, said external connector being externally connectable to a transcutaneous catheter.

2. An extracorporeal tubing set as claimed in claim 1, wherein the access tube and the external connector are releasably attached one to another in series at the distal end of the fluid transfer tube.

3. An extracorporeal tubing set as claimed in claim 2, wherein a distal-most one of the access tube and the external connector is removable from the fluid transfer tube to permit a proximal-most one of the access tube and the external connector to be exposed, so as to enable the exposed one of the access tube and external connector to be connected to one of the implanted port and the transcutaneous catheter.

4. An extracorporeal tubing set as claimed in claim 3, wherein the access tube is distal to the external connector.

5. An extracorporeal tubing set as claimed in claim 1, wherein the access tube and external connector are attached in parallel to the distal end of the tube.

6. An extracorporeal tubing set as claimed in claim 1, wherein at least the access tube and the external connector are packaged in a sterile condition.

7. An extracorporeal tubing set as claimed in claim 1, in which the fluid transfer tube comprises a distal tube portion defining said distal end, and two proximal tube portions connected in fluid flow communication with the distal tube portion, each proximal tube portion defining a proximal end of the fluid transfer tube.

8. An extracorporeal tubing set as claimed in claim 7, in which the proximal ends are connectable to dialysis solution containers.

9. A dialysis tubing set comprising:
a fluid transfer tube having a proximal end and a distal end, wherein the proximal end is connectable to a source of dialysis solution;
an access tube connected, or connectable, to the distal end of the fluid transfer tube, said access tube percutaneously connectable to an implanted port; and
an external connector connected, or connectable, to the distal end of the fluid transfer tube, said external connector externally connectable to a transcutaneous catheter.

10. A dialysis tubing set as in claim 9, wherein the access tube and external connector are releasably attached one to another in series at the distal end of the fluid transfer tube.

11. A dialysis tubing set as in claim 10, wherein a distal-most one of the access tube and external connector is removable from the fluid transfer tube to permit a proximal-most one of the access tube and external connector to be connected to one of the implanted port and transcutaneous catheter.

12. A dialysis tubing set as in claim 11 wherein the access tube is distal to the external connector.

13. A dialysis tubing set as in claim 9, wherein the access tube and external connector are attached in parallel to the distal end of the fluid transfer tube.

14. A dialysis tubing set as in claim 9, wherein at least the access tube and the external connector are packaged in a sterile condition.

15. A dialysis tubing set as in claim 9, in which the fluid transfer tube comprises a distal tube portion defining said distal end, and two proximal tube portions connected in fluid flow communication with the distal tube portion, each proximal tube portion defining a proximal end of the fluid transfer tube.

16. A dialysis tubing set as in claim 15, in which the proximal ends are each connectable to a dialysis solution container.

17. An access tube set for adapting at least one tubing set, having an external connector normally connectable to a specific complementary connector of a transcutaneous catheter, so as to render the tubing set operatively connectable with an implanted subcutaneous port, the access tube set comprising:
an access tube connectable with the implanted subcutaneous port; and
a connector operatively connected to the access tube, the connector being complementary to the external connector of the tubing set, so that the connector of the access tube set can be connected to the external connector of the tubing set thereby to render the tubing set selectively connectable to the implanted port.

18. An adapter kit for adapting any one of a plurality of different tubing sets, each having a specific external connector normally connectable to a specific complementary external connector of a transcutaneous catheter, so as to render any one of the plurality of different tubing sets selectively connectable with an implanted subcutaneous port, the adapter kit comprising:
an access tube set having an access tube connectable with the implanted subcutaneous port and a connector operatively connected to the access tube, the connector being complementary to the external connector of a specific one of the different tubing sets so that the access tube set can be connected to that external connector to render that tubing set operatively connectable to the implanted port; and
at least one adapter comprising a first connector, releasably connectable to the connector of the access tube set, and an opposed connector connected to the first connector, the opposed connector being complementary to the external connector of another specific one of the different tubing sets, the adapter being mountable on the connector of the access tube set thereby to enable the access tube set to be connected to the external connector of the other specific one of the different tubing sets thereby to render the other specific one of the different tubing sets operatively connectable to the implanted port.

19. An adapter kit as claimed in claim 18, which comprises a plurality of adapters each comprising a first connector, releasably connectable to the connector of the access tube set, and an opposed connector connected to the first connector, the opposed connectors of each adapter being complementary to the external connector of a different one of the different tubing sets.

20. An adapter kit as claimed in claim 19, which comprises a plurality of adapters, each comprising a first connector and an opposed connector complementary to the external connector of a different specific tubing set, the first connector of one adapter being complementary to the opposed connector of another adapter so that the adapters can be connected one to another in series and to the connector of the access tube set.

21. An access tube set comprising:
an access tube arranged to access a patient body; and
at least two different connectors operatively connected, or connectable, to the access tube, each connector being complementary to a different conventional connector of a different conventional tubing set, so as to render the access tube selectively connectable to any one of the different conventional tubing sets.

22. An access tube set as claimed in claim 21, wherein a first of the at least two connectors is complementary to a conventional connector on a hemodialysis tubing set, and a second of the at least two connectors is complementary to a non-conventional connector of a non-conventional hemodialysis tubing set, the second connector having an internal diameter greater than that of the first connector.

23. An access tube set as claimed in claim 22, wherein the first connector is in the form of a Luer type connector.

24. An access tube set as claimed in claim 23, wherein the first connector is in the form of a female Luer type connector.

25. An access tube set as claimed in claim 23, wherein the first connector is in the form of a male Luer type connector.

26. An access tube set as claimed in claim 22, wherein the second connector is connected to the access tube, and the access tube set further comprises an adapter defining the first connector, the adapter being releasably mountable on the second connector.

27. An access tube set as claimed in claim 21, wherein each connector is complementary to a different connector of different peritoneal dialysis tubing sets, which connectors are arranged for extracorporeal connection to transcutaneous catheters.

28. An access tube set as claimed in claim 27, wherein one of the connectors is connected to the access tube, and the access tube set further comprises an adapter defining the other connector, the adapter being releasably mountable on the connector connected to the access tube.

29. An access tube set comprising:
an access tube arranged to access a patient body; and
at least two different connectors operatively connected, or connectable, to the access tube, each connector being complementary to a different conventional connector of a different conventional tubing set, so as to render the access tube selectively connectable to any one of the different conventional tubing sets.

30. A tubing set as claimed in claim 29, wherein a first of the at least two connectors is complementary to a conventional connector on a hemodialysis patient access device, and a second of the at least two connectors is complementary to a non-conventional connector of a non-conventional hemodialysis patient access device, the second connector having an internal diameter greater than that of the first connector.

31. A tubing set as claimed in claim 30, wherein the first connector is in the form of a Luer type connector.

32. A tubing set as claimed in claim 31, wherein the first connector is in the form of a female Luer type connector.

33. A tubing set as claimed in claim 31, wherein the first connector is in the form of a male Luer type connector.

34. A tubing set as claimed in claim 30, wherein the second connector is connected to the fluid transfer tube, and the tubing set further comprises an adapter defining the first connector, the adapter being releasably mountable on the second connector.

35. A tubing set as claimed in claim 29, wherein each connector is complementary to a different connector of different peritoneal dialysis catheters.

36. A tubing set as claimed in claim 35, wherein one of the connectors is connected to the fluid transfer tube, and the tubing set further comprises an adapter defining the other connector, the adapter being releasably mountable on the connector connected to the access tube.

37. An access device, for accessing a patient body to perform a fluid transfer procedure, the access device comprising:
an accessing portion arranged to access a patient body; and
at least two connectors operatively connected, or connectable, with the accessing portion, each connector being complementary to a different connector of different tubing sets so as to render the access device selectively coupleable to any one of the different tubing sets.

38. An access device as claimed in claim 37, wherein a first of the at least two connectors is complementary to a conventional connector on a hemodialysis tubing set, and a second of the at least two connectors is complementary to a non-conventional connector of a non-conventional hemodialysis tubing set, the second connector having an internal diameter greater than that of the first connector.

39. An access device as claimed in claim 38, wherein the first connector is in the form of a Luer type connector.

40. An access device as claimed in claim 39, wherein the first connector is in the form of a female Luer type connector.

41. An access device as claimed in claim 39, wherein the first connector is in the form of a male Luer type connector.

42. An access device as claimed in claim 38, wherein the second connector is connected to the accessing portion, and the access device further comprises an adapter defining the first connector, the adapter being releasably mountable on the second connector.

43. An access device as claimed in claim 37, wherein each connector is complementary to a different connector of different peritoneal dialysis tubing sets, which tubing sets are normally connectable to transcutaneous catheters.

44. An access device as claimed in claim 43, wherein one of the connectors is connected to the accessing portion, and the access device further comprises an adapter defining the other connector, the adapter being releasably mountable on the connector connected to the accessing portion.

45. An access device as claimed in claim 37, in which the accessing portion is in the form of an access tube for transcutaneously accessing a subcutaneously implanted port in a patient's body.

46. An access device as claimed in claim 37, wherein the accessing portion is in the form of part of a catheter arranged to be implanted transcutaneously in a patient's body.
